(19) United States
(12) Patent Application Publication
(10) Pub. No.: US 2024/0374871 A1
(43) Pub. Date: Nov. 14, 2024

(54) TWISTED GUIDEWIRE

(71) Applicant: Abbott Cardiovascular Systems Inc., Santa Clara, CA (US)

(72) Inventors: Puneet Kamal Singh Gill, Anaheim, CA (US); Jonathan Durcan, Temecula, CA (US); Joshua Jensen, Canyon Lake, CA (US)

(73) Assignee: Abbott Cardiovascular Systems Inc., Santa Clara, CA (US)

(21) Appl. No.: 18/196,283

(22) Filed: May 11, 2023

(51) Int. Cl. A61M 25/09

(52) U.S. Cl. CPC A61M 25/09; A61M 2025/09191

(57) ABSTRACT

A guidewire having improved torque characteristics in a proximal section of the guidewire. The proximal section has a square cross-section and a plurality of twists along the length of the proximal section. The plurality of twists have a pitch in a range from 1 to 3 twists per 1.0 inch so that the guidewire has less than a 15° torque delay when the proximal section is subjected to torsional forces

### BACKGROUND

The technology herein relates to the field of guidewires for advancing intraluminal devices such as stent delivery catheters, balloon dilatation catheters, atherectomy catheters and the like within body lumens.

In general, guidewires can be torqued to facilitate navigation through tortuous vessels and to facilitate therapeutic procedures, such as the placement of balloon dilation catheters and compatible stent devices, in arteries and veins within, but not limited to, aortic, iliac, femoral, popliteal and infra-popliteal regions. In a typical coronary procedure a guiding catheter having a preformed distal tip is percutaneously introduced into a patient's artery, e.g., femoral or brachial artery, by means of a conventional Seldinger technique and advanced therein until the distal tip of the guiding catheter is seated in the ostium of a desired coronary artery. There are two basic techniques for advancing a guidewire into the desired location within the patient's coronary anatomy, the first is a preload technique which is used primarily for over-the-wire (OTW) devices and the second is a bare wire technique which is used primarily for rapid exchange type systems. With the preload technique, a guidewire is positioned within an inner lumen of an OTW device such as a dilatation catheter or stent delivery catheter with the distal tip of the guidewire just proximal to the distal tip of the catheter and then both are advanced through the guiding catheter to the distal end thereof. The guidewire is first advanced out of the distal end of the guiding catheter into the patient's coronary vasculature until the distal end of the guidewire crosses the arterial location where the interventional procedure is to be performed, e.g., a lesion to be dilated or a dilated region where a stent is to be deployed. The catheter, which is slidably mounted onto the guidewire, is advanced out of the guiding catheter into the patient's coronary anatomy over the previously introduced guidewire until the operative portion of the intravascular device, e.g., the balloon of a dilatation or a stent delivery catheter, is properly positioned across the arterial location. Once the catheter is in position with the operative means located within the desired arterial location, the interventional procedure is performed. The catheter can then be removed from the patient over the guidewire. Usually, the guidewire is left in place for a period of time after the procedure is completed to ensure reaccess to the arterial location.

With the bare wire technique, the guidewire is first advanced by itself through the guiding catheter until the distal tip of the guidewire extends beyond the arterial location where the procedure is to be performed. Then a rapid exchange (RX) catheter is mounted onto the proximal portion of the guidewire which extends out of the proximal end of the guiding catheter, which is outside of the patient. The catheter is advanced over the guidewire, while the position of the guidewire is fixed, until the operative means on the RX catheter is disposed within the arterial location where the procedure is to be performed. After the procedure, the intravascular device may be withdrawn from the patient over the guidewire or the guidewire advanced further within the coronary anatomy for an additional procedure.

Conventional guidewires for angioplasty, stent delivery, atherectomy and other vascular procedures usually comprise an elongated core member with one or more tapered sections near the distal end thereof and a flexible body such as a helical coil or a tubular body of polymeric material disposed about the distal portion of the core member. A shapeable member, which may be the distal extremity of the core member or a separate shaping ribbon, which is secured to the distal extremity of the core member, extends through the flexible body and is secured to the distal end of the flexible body by soldering, brazing or welding which forms a rounded distal tip. Torqueing means may be provided on the proximal end of the core member to rotate, and thereby steer, the guidewire while it is being advanced through a patient's vascular system.

It is typical that best medical practice for anatomical insertion requires a guidewire that has behavioral characteristics that vary along its length. For example, under some conditions, the distal end of the guidewire may be required to be more flexible than the proximal end so that the distal end may more easily be threaded around the more tortuous distal branches of the luminal anatomy. Further, the proximal end of the guidewire may be required to have greater torsional stiffness than the distal end because, upon rotation of the guidewire, the proximal end must carry all the torsional forces that are transmitted down the length of the guidewire, including what is required to overcome cumulative frictional losses.

For certain procedures, such as when delivering stents around a challenging take-off, e.g., a shepherd's crook, tortuosities or severe angulation, substantially more support and/or vessel straightening is frequently needed from the guidewire than normal guidewires can provide. Guidewires have been commercially introduced for such procedures which provide improved distal support over conventional guidewires, but such guidewires are not very steerable and in some instances are so stiff that they can damage vessel linings when advanced therethrough. What has been needed and heretofore unavailable is a guidewire which provides a high level of distal support by providing a high degree of torque along the proximal section of the guidewire.

### SUMMARY OF THE INVENTION

An important aspect of the present invention is to provide a guidewire having superior torque characteristics while also providing reduced friction between the guidewire and the delivery system. The present invention guidewire increases bending stiffness and also enhances proximal gripping for navigating tortuous vessels.

In one embodiment, a guidewire comprises an elongated wire having a proximal end and a distal end. The length of the guidewire can vary significantly based on numerous factors such as where the guidewire is inserted into the body. In one embodiment, a proximal section of the guidewire has a length in a range from 12 inches to 120 inches and a distal section has a length in a range from zero inches to 8.0 inches. At least a portion of the proximal section has a square transverse cross-section with four rounded corners. The proximal section has a plurality of twists with the pitch being in a range of 1 to 3 twists per 1.0 inch. When subjected to torsional forces at the proximal section, the distal section has less than a 15° torque delay.

In another embodiment, the guidewire is structurally the same as disclosed supra, with the exception that the proximal section has a plurality of twists with the pitch being no more than 2.0 twists per 1.0 inch. When subjected to torsional forces at the proximal section, the distal section has less than 15° of rotation torque delay.

In all of the embodiments disclosed herein, the torque delay in the distal section can range from 0° rotation to 35° rotation and still provide excellent torque response.

In one embodiment, a guidewire is formed by an elongated wire having a proximal end and a distal end. A proximal section extends from the proximal end toward the distal end and has a length in a range from 12 inches to 120 inches. A distal section extends from the distal end toward the proximal end and has a length in a range from zero inches to 8.0 inches. The proximal section has a square transverse cross-section with four corners. The four corners are preferably rounded and had a radius in a range from 0.010 to 0.014 inch., and preferably a radius of 0.012 inch. The proximal section has a plurality of twists with the pitch of the plurality of twists being 2.0 twists per 1.0 inch. When subjected to torsional forces, the distal section has less than a 15° torque delay.

### BRIEF DESCRIPTION OF THE DRAWINGS

FIG. 1 is an elevational view depicting the guidewire of the present invention having a square cross-section proximal section and a round cross-section distal section.

FIG. 2 is an elevational view of the guidewire of the present invention depicting twists formed in a proximal section of the guidewire.

FIG. 3 is a transverse cross-sectional view taken along lines 3-3 of the guidewire in FIG. 1 depicting a square cross-section having rounded corners.

FIG. 4 is a graph depicting clockwise torque test data resulting from testing on several commercial guidewires compared to the present invention guidewire.

FIG. 5 is a graph depicting counterclockwise torque test data resulting from testing on several commercial guidewires compared to the present invention guidewire.

FIG. 6 is a graph depicting clockwise and counterclockwise test data resulting from testing on a commercial guidewire compared to the present invention guidewires.

### DETAILED DESCRIPTION OF THE PREFERRED EMBODIMENTS

The guidewires of the present invention provide enhanced torque characteristics, higher bending stiffness as compared to round wires, increased tactile feel for physicians, and reduced friction between the guidewire and the catheter.

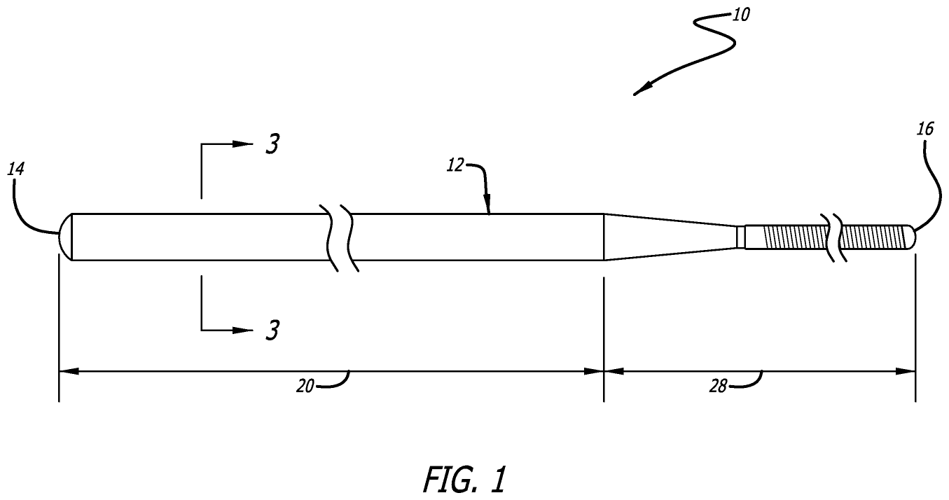

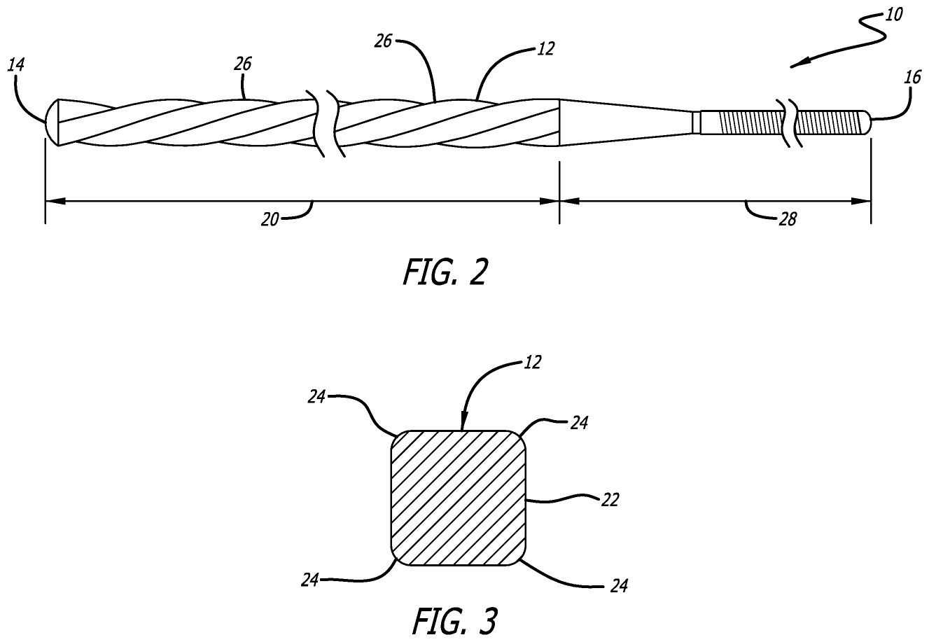

In keeping with the invention, as shown in FIGS. 1-6, a guidewire 10 is formed from an elongated wire 12 having a proximal end 14 and a distal end 16. The elongated wire has an overall length 18 in a range from 20 inches (50.8 cm) to 120 inches (304.8 cm), depending on factors such as the location on the human body where the guidewire 10 is inserted. In this embodiment, a proximal section 20 has a length preferably in a range from 12 inches (30.5 cm) to 71 inches (180.3 cm), and more preferably in a range from 40 inches (101.6 cm) to 60 inches (152.4 cm). In one embodiment, the proximal section is 78.7 inches (200 cm). The proximal section 20 has a transverse square cross-section 22 with four corners 24, wherein the four corners 24 are rounded. The four corners 24 are rounded to a radius in a range from 0.005 inch (0.127 mm) to 0.03 inch (0.762 mm). In one embodiment, the four corners 24 are rounded to 0.0108 inch (0.2743 mm).

The proximal section 20 has a plurality of twists 26 with the pitch of the twists 26 in a range from 1 to 3 twists per 1.0 inch (25.4 mm). In one embodiment, the proximal section 20 has no more than 2 twists per 1.0 inch (25.4 mm). In another preferred embodiment, the proximal section 20 has 2 twists 26 per 1.0 inch. When the proximal section 20 is subjected to torsional forces (described more fully herein), a distal section 28 has a torque delay in range from 0° to 35°. Preferably, the torque delay in the distal section 28 is in a range from 0° to 15°. In one embodiment, the torque delay is the distal section 28 is less than 15°. The distal section 28 has a length 30 in a range from 2.0 inches (5.08 cm) to 8.0 inches (20.32 cm). In one embodiment, the length of one pitch or twist 26 is in a range from 0.2 inch to 1.0 inch, and preferably the pitch length is 0.5 inch.

Guidewire lengths are well known in the art and can range from 180 cm to 300 cm for coronary artery applications, to much shorter lengths for other applications. Importantly, the proximal section 20 may be substantially longer than the distal section 28 of the elongated wire 12. For example, for a standard 70.87 inches (180 cm) long guidewire 10, the proximal section 20 can range from 37.40 inches (95 cm) to 70.87 inches (180 cm) and preferably range from 64.96 inches (165 cm) to 68.90 inches (175 cm).

The guidewire 10 preferably is formed from any superelastic or linear elastic material known in the art, and more preferably formed from nitinol. Guidewire 10 can be formed from other metal alloys including stainless steel, titanium, and cobalt chromium.

A polymer cover (not shown) can be formed by any polymer well known in the art for use with guidewires, catheters, and stents. The elongated member 12 is optionally coated with a lubricious coating such as a fluoropolymer, e.g., TEFLON®, which extends the length of the proximal core section. Hydrophilic coatings may also be employed. The diameter of the guidewire 10 may be varied to suit the particular procedures in which it is to be used and the materials from which it is constructed. The guidewire diameter generally ranges from about 0.008 inch to about 0.035 inch (0.203 mm to 0.889 mm), more typically ranging from about 0.012 inch to about 0.018 inch (0.305 mm to 0.547 mm), and preferably about 0.014 inch (0.336 mm) for coronary anatomy and 0.018 inch (0.547 mm) and 0.035 inch (0.889 mm) for peripheral anatomy.

The guidewire 10 disclosed herein can be formed in a number of configurations. For example, as shown in FIGS. 1-3, the proximal section 20 can be formed from stainless steel and have a square transverse cross-section (FIG. 3). The stainless steel proximal section 20 is solid state welded to the distal section 28, which has a round transverse cross-section. The distal section 28 is formed from nitinol (NiTi) or similar superelastic material known in the art. The four corners 24 on the square cross-section proximal section 20 are rounded, generally by grinding as is known in the art. The radius on the rounded corners 24 can vary as set forth herein.

The proximal end 14 of the proximal section 20 is secured in a machine to impart the disclosed number of twists 26 per unit length. The proximal section 20 is rotated by the machine while a distal end of the proximal section is held stationary, thereby forming the twists 26 in the proximal section. Depending on the twist pattern and diameter of the guidewires, a grinding wheel having a width of 0.5 inch to 2 inches can be used to further form the twists 26 and to round the four corners 24. The twist pattern is imparted to the proximal section 20 only. In one embodiment, a number of twists 26 are formed along a length of the proximal section 20, but not the entire length. The twists 26 can be alternated with non-twisted portions of the proximal section. Further, the number of twists 26 per unit length can be different along different sections of the proximal section 20. In one embodiment, the twists 26 are formed in the proximal section 20 before the proximal section 20 is welded to the distal section 28.

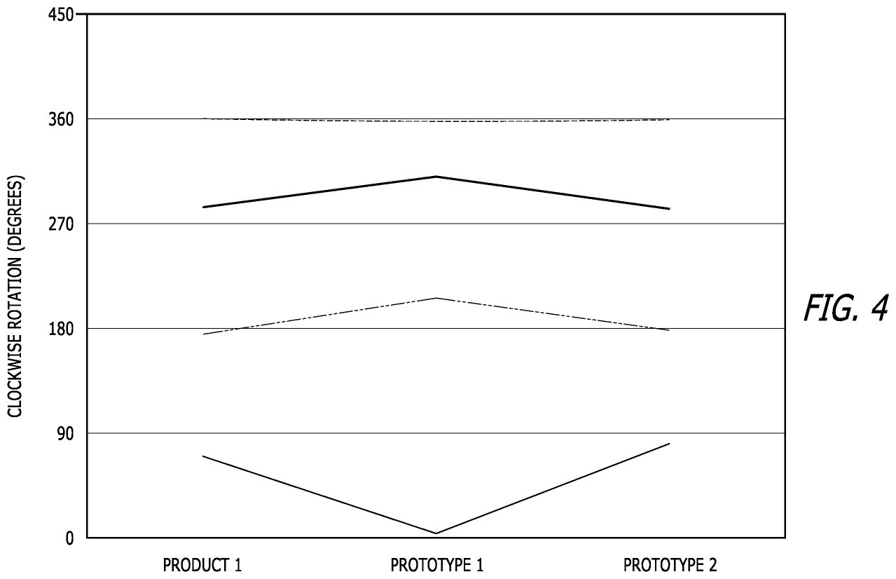

Several prototype guidewires 10 of the embodiments disclosed herein were tested for torque delay as compared to a commercially available guidewire having a twisted pattern. Ideally, a physician would want a guidewire having a 1:1 ratio torque response, i.c., 90° of rotation on the proximal end results in 90° rotation on the distal end. Thus, there is 0° torque delay in the distal end. As shown in FIGS. 4-6, torque test data was collected for Prototype 1 and Prototype 2 of the present invention (having a square cross-section) compared to Product 1 (a commercially available guidewire having a round cross-section). Differences in the results of the test data depend on many factors for the tested guidewire such as the cross-section shape, metal alloy, length, diameter, and location and number of twists per unit length. The Prototypes 1 and 2 have a twisted square cross-section in the proximal section while Product 1 has a round cross-section core. Thus, the test data is presented for comparison only.

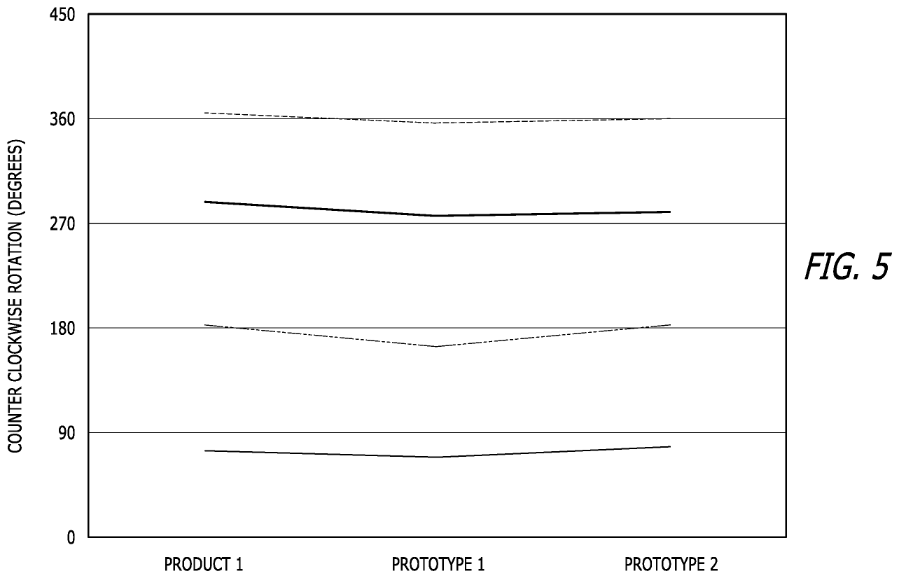

The tests were performed by inserting the proximal end of the guidewire into a machine that firmly grips the proximal end. The guidewire is stretched out with a video camera focused on the distal end. The machine then rotates the proximal end 90° in either a clockwise or counterclockwise direction, and the system is paused a few seconds to revive any buildup, and then the distal end is observed using the video camera to determine how much torque delay (rotation) occurred at the distal end. After observing the torque delay in degrees at the distal end 16 of the guidewire, the machine rotated the guidewire clockwise (FIG. 4) or counterclockwise (FIG. 5) another 90° (180° total), and again the distal end was observed by video camera for torque delay. The same process was repeated at 270° and 360° of rotation at the proximal end of the guidewires.

As shown in FIGS. 4 and 5, the rotation of the proximal end in degrees is on the Y-axis and the guidewires tested are labeled on the X-axis. As show in FIG. 4, at 90° of clockwise rotation applied to the proximal end, the Prototype 2 guidewire had a very low torque delay response of approximately 4° to 15°, while the Prototype 1 and Product 1 guidewires had relatively higher torque delay compared to Prototype 2. Ideally, a torque delay in the range of 0° to 35° is desirable, and a torque delay in the range of 0° to 15° is more preferable. FIG. 5 shows similar results when the guidewires are rotated or torqued in a counterclockwise direction. Differences in torque delay data between the clockwise and counterclockwise rotation at the proximal end can be explained in part by the direction of the twists on the tested wires.

As shown in FIGS. 4 and 5, at 180° of rotation at the proximal end of the guidewires, the Prototype 2 and Product 1 guidewires had similar good torque delay results, while Prototype 1 guidewire shows energy buildup and higher torque delay at the distal end 16, which is undesirable as it is difficult to predict how the distal section 28 and the distal end 16 are going to perform. At 360° of rotation at the proximal end 20 of the tested guidewires, the distal end 16 of all of the tested guidewires were able to catch up and there was 0° of torque delay.

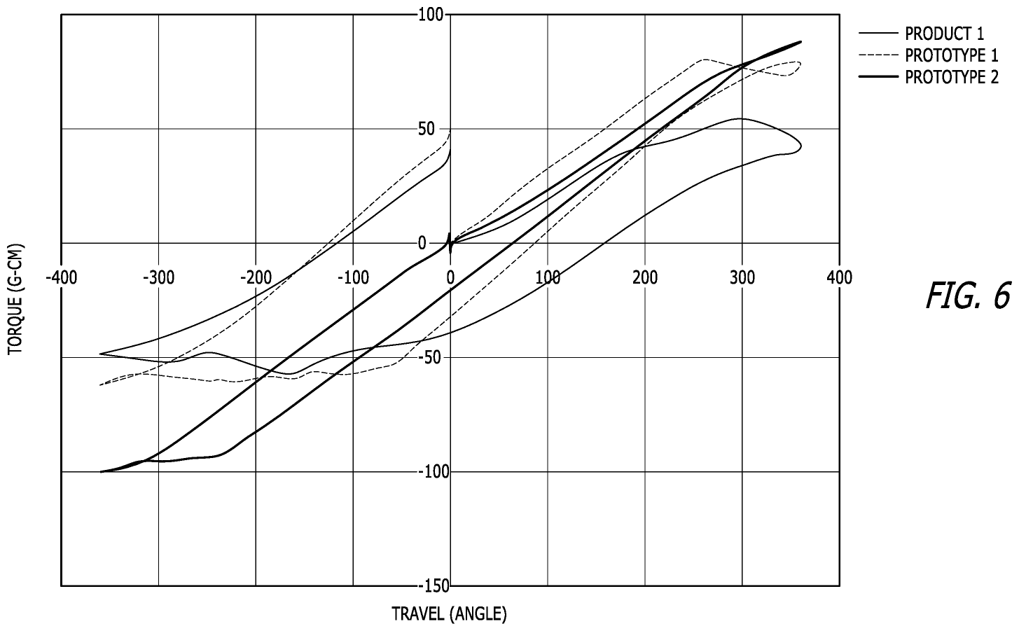

A variability chart is shown in FIG. 6 representing the travel (in degrees) of the distal end of the guidewires. FIG. 6 is a graphic display of the data generated where the distal end of the guidewire is passed through a simulated fixture and a tip of the guidewire is held in a chuck with a sensor attached. The proximal end of the guidewire is torqued 360° in a clockwise direction, back to a neutral position, and then torqued 360° in a counter clockwise direction. The sensor measures the torque delay at the distal tip of the guidewire.

While the invention has been illustrated and described herein in terms of its use as a guidewire, it will be apparent to those skilled in the art that the guidewire can be used in all vessels of the body. All dimensions disclosed herein are by way of example. Other modifications and improvements may be made without departing from the scope of the invention.

I claim:

1. A guidewire, comprising:
an elongated wire having a proximal end and a distal end;
a proximal section having a length in a range from 12 inches to 120 inches and a distal section having a length in a range from zero inches to 12 inches;
the proximal section having a transverse square cross-section with four corners, the four corners being rounded;
the proximal section having a plurality of twists with the pitch of the plurality of twists being in a range of 1 to 3 twists per 1.0 inch; and
the distal section having less than a 15° torque delay when the proximal section is subjected to torsional forces.

2. The guidewire of claim 1, wherein the guidewire has a diameter in a range from 0.014 inch to 0.035 inch.

3. The guidewire of claim 2, wherein the elongated wire has a length of 120 inches.

4. The guidewire of claim 2, wherein the elongated wire has a length of 71 inches.

5. The guidewire of claim 2, wherein the rounded four corners have a radius in a range from 0.005 inch to 0.127 inch.

6. A guidewire, comprising:
an elongated wire having a proximal end and a distal end;
a proximal section having a length in a range from 12 inches to 120 inches and a distal section having a length in a range from zero inches to 12 inches;
the proximal section having a transverse square cross-section with four corners, the four corners being rounded;
the proximal section having a plurality of twists with the pitch of the plurality of twists being no more than 2 twists per 1.0 inch; and
the distal section having less than a 15° torque delay when the proximal section is subjected to torsional forces.

7. The guidewire of claim 6, wherein the guidewire has a diameter in a range from 0.014 inch to 0.035 inch.

8. The guidewire of claim 7, wherein the elongated wire has a length of 120 inches.

9. The guidewire of claim 7, wherein the elongated wire has a length of 71 inches.

10. The guidewire of claim 7, wherein the rounded four corners have a radius in a range from 0.005 inch to 0.127 inch.

11. A guidewire, comprising:
an elongated wire having a proximal end and a distal end;
a proximal section having a length in a range from 12 inches to 120 inches and a distal section having a length in a range from zero inches to 12 inches;
the proximal section having a transverse square cross-section with four corners, the four corners being rounded;
the proximal section having a pitch of 2 twists per 1.0 inch; and
the distal section having less than a 15° torque delay when the proximal section is subjected to torsional forces.

12. The guidewire of claim 11, wherein the guidewire has a diameter in a range from 0.014 inch to 0.035 inch.

13. The guidewire of claim 12, wherein the elongated wire has a length of 120 inches.

14. The guidewire of claim 12, wherein the elongated wire has a length of 71 inches.

15. The guidewire of claim 12, wherein the rounded four corners have a radius 0.005 inch to 0.127 inch.
